(19) United States
(12) Patent Application Publication
(10) Pub. No.: US 2006/0216269 A1
(43) Pub. Date: Sep. 28, 2006

(54) Dendritic cell tumor injection (DCTI) therapy

(76) Inventor: Kenichiro Hasumi, Suginami-ku (JP)

Correspondence Address: Eric A. Lerner;Eckert Seamans Cherin & Mellott, LLC, 44th Floor, 600 Grant Street, Pittsburgh, PA, 15219, US

(21) Appl. No.: 11/227,374

(22) Filed: Sep. 15, 2005

Related U.S. Application Data
(60) Provisional application No. 60/610,822, filed on Sep. 17, 2004.

(51) Int. Cl. A61K 48/00; A61K 38/20; A61K 38/19; A61K 31/739; A61K 36/06

(52) U.S. Cl. 424/85.1; 424/85.2; 424/93.21; 435/456; 424/195.15; 514/54

(57) ABSTRACT

The invention relates to a method of treating tumor cells within a patient wherein immature dendritic cells developed from the patient's monocyte cells and an adjuvant are introduced into the patient directly into the patient's tumor cells. The immature dendritic cells and adjuvant combine with the antigens in the tumor cells to form a cancer vaccine, thereby immediately treating the tumor cells of the patient. The invention also provides a precursor treatment step of treating the patient with a radiation therapy or chemotherapy regimen.

### CROSS REFERENCE TO RELATED APPLICATION

The present application claims priority from provisional application 60/610,822 filed Sep. 17, 2004.

### FIELD OF THE INVENTION

The present invention relates to a tumor therapy that includes the injection of immature dendritic cells and adjuvant directly into the patient's (a human or an animal) tumor tissue, which presents antigenicity as a vaccine antigen at the injection sight. Conjugation of these elements within the tumor tissue rapidly induce and activate the patient's immune system to dramatically reduce and/or eliminate tumor cells. Most adjuvants, which augment the immune response, can be directly injected with immature dendritic cells to the tumor tissue to achieve the reduction or elimination of tumor tissues.

### BACKGROUND INFORMATION

Immunological adjuvants are used in combination with vaccines to augment the immune response to the antigen. One way in which immunological adjuvants function is by attracting macrophages to the antigen, so that the macrophages can present the antigen to the regional lymph nodes and initiate an effective antigenic response. Adjuvants may also act as carriers themselves for the antigen, or may influence the immune response by other mechanisms such as depot effect, cytokine induction, complement activation, recruiting of different cell populations of the immunological system, antigen delivery to different antigen presenting cells, regulation of the expression of HLA class I or class II molecules and the stimulation to produce different antibody subtypes. Many of the newer vaccines are only weakly immunogenic and thus require the presence of adjuvants.

Materials having adjuvant activity are well known. Alum (Al(OH)₃), and similar aluminum gels are adjuvants licensed for human use. The adjuvant activity of alum was first discovered in 1926 by Glenny (Chemistry and Industry, Jun. 15, 1926; J. Path. Bacteriol, 34, 267). Aluminum hydroxide and aluminum phosphate (collectively commonly referred to as alum) are routinely used as adjuvants in human and veterinary vaccines. The efficacy of alum in increasing antibody responses to diphtheria and tetanus toxoids is well established and, more recently, a HBsAg vaccine has been adjuvanted with alum.

One line of research in the development of adjuvants has been directed to the study of dendritic cells. Dendritic cells (DC) are professional antigen presenting cells (APC) that have the unique capacity to initiate primary immune responses in vivo and in vitro. They are derived from myeloid (DC1) or lymphoid (DC2) precursors and are distributed in their immature form throughout the body in tissues that commonly encounter environmental pathogens (skin, mucus membranes, gut epithelia, etc.). Whereas DC1 and DC2 comprise a small percentage of the total number of mononuclear cells in the peripheral circulation, DC1 precursors in the form of CD14+/CD11c+/HLA-DR+monocytes are relatively abundant, constituting about 10% to 15% of mononuclear blood cells.

Immature DC express a host of surface structures that are involved in antigen acquisition, DC activation/maturation, and antigen presentation. Once DC encounter antigen, they undergo a maturation process characterized by the up-regulation of HLA class I and II molecules as well as co-stimulatory molecules and interact with cognate receptors on T and B lymphocytes, resulting in the generation of antigen specific cellular and humoral immune responses.

DC are considered to be the primary APC in the immune system. The ability to isolate these cells and/or their precursors and to study them in vitro has added considerable dimension to knowledge of their role in innate and acquired immunity. The classic means of generating human DC in vitro is to isolate and enrich CD14+-monocytes from peripheral blood and culture them for various periods of time in GM-CSF and IL-4 followed by final maturation with a number of cytokines, including IL-2, IL-6, IL-7, IL-13, IL-15, TNF, IL-10, or with various other agents including lipopolysaccharides, PGE₂, type 1 interferons, or double-stranded RNA.

Numerous investigators have shown that these in vitro generated monocyte-derived DC are potent antigen presenting cells (APC) capable of initiating primary and recall antigen-specific CD4+ and CD8+ T cell responses. Recent in vitro studies have generated a rather extensive body of information regarding the biology of DC1 and shed light on the processes whereby antigen specific immune responses are generated in vivo. In the peripheral tissues, immature DC acquire antigenic materials in the context of danger signals initiating a complex cytokine/chemokine milieu that is generated by DC and other cell types in the vicinity. Soluble mediators produced by DC may act in an autocrine or paracrine fashion. T cells produce additional cytokines and chemokines following interaction with antigen armed DC, as do other immune cells that are activated by the cytokines released. This complex network of interactions may in turn create an environment that promotes the generation of DC from their monocyte precursors.

It is thought that those adjuvants which promote that maturation of dendritic cells, when administered in combination with a vaccine antigen, will result in more antigen presenting cells presenting the vaccine antigen to T lymphocytes and B cells, thus bolstering the immune response to the vaccine antigen. However, isolation of the most effective vaccine antigen has been extremely difficult since antigenicity of APC has always been subject to its evolution with antigenic drift and/or shift, and therefore many of the newer vaccines are only weakly immunogenic even though dendritic cells and adjuvant are present. The most effective vaccine antigen against the live tumor cells should be used with dendritic cells and adjuvant during a course of treatment to promote and to induce a rather strong immunogenicity.

### DESCRIPTION OF INVENTION

The present invention solves the above need by providing the most effective antigenic vaccine antigen with dendritic cells and adjuvant to increase the amount and quality of the immune response against tumor cells.

The present invention provides treatment tumor tissue using full antigenic elements, which include antigenicity of both known and unknown antigen presenting cells, by locating them within the live tumor tissue in the human body (or alternatively, the body of an animal). This is in contrast to prior art cultured antigens obtained from tumor cell lines or any process added antigen, which have limited antigencity and outdated antigenic data or potency as a vaccine antigen for the patient's tumor cells. In particular, the present invention relates to a therapy that includes the injection of immature dendritic cells and adjuvant directly into the patient's tumor tissue, which presents antigenic elements as the vaccine antigen at the injection sight. The conjugation of these elements within the tumor tissue rapidly induce and activate the patient's immune system to dramatically reduce and/or eliminate tumor cells. Most adjuvants, which augment the immune response, can be directly injected with immature dendritic cells into the tumor tissue to achieve the reduction or elimination of tumor cells. Such adjuvants may include, without limitation, lipid-based, protein-based and polysaccharides-based adjuvants, such as

Marignase

LCM

Agaricus

OK432

BCG

Lentinan (shiitake)

Reishi

Sarunokoshikake

TNF

Meshimakobu

Froint's comlete or imcomplete adjuvant

LPS

Fatty acids

Phospholipids

Cytokines

Virus.

The present invention provides rapid reduction and/or elimination of tumor cells, which can be visually detected by MRI and/or CT and/or Echo scan within two weeks after the injection. The therapy according to a preferred embodiment of the invention includes the following steps:
Step 1: Colleting peripheral blood monocyte cells (PBMC) from a patient
Step 2: Culturing these PBMC with GM-CFS and IL-4 to immature dendritic cells.

Step 3: Injecting the cultured immature dendritic cells and an adjuvant into the tumor. Step 4: Evaluating the tumor in two weeks Step 1: Collecting PBMC from Patient Step 2: Culturing PBMC to immature DC with IL-4 GM-CS Step 3: Injecting immature DC and Adjuvant to the tumor Step 4: Evaluating the tumor in two weeks

Step 1: Collecting PBMC from Patient
Step 2: Culturing PBMC to immature DC with IL-4 GM-CS
Step 3: Injecting immature DC and Adjuvant to the tumor
Step 4: Evaluating the tumor in two weeks

In one particular embodiment, the effectiveness (immuno-response) of this method of treatment can be enhanced by pre-treating the tumor cells using known chemotherapy and/or radiation therapy techniques, which diminish the existing immune system, prior to the steps 1-4 described above. In addition, the effectiveness (immuno-response) of this method of treatment can also be enhanced by injecting the tumors cells with an anti T-cell monoclonal antibody prior to the steps 1-4 described above (either alone or in addition to the chemotherapy and/or radiation therapy described above).

I claim:

1. A method of reduction of tumor cells in tumor tissue of a patient comprising the steps of:
collecting monocyte cells from the patient,
culturing the monocyte cells with one or a plurality of factors to form immature dendritic cells from the monocyte cells,
introducing the immature dendritic cells into the tumor tissue of the patient, and
introducing an adjuvant into the tumor tissue of the patient.

2. The method of claim 1, wherein the patient is a human.

3. The method of claim 1, wherein the said one or a plurality of factors are selected from the group consisting of: IL-4 and GM-CFS.

4. The method of claim 1, wherein the adjuvant is selected from the group consisting of: marignase, LCM, agaricus, OK432, BCG, lentinan, Reishi, Sarunokoshikake, TNF, Meshimakobu, Froint's complete or incomplete adjuvant, LPS, fatty acids, and phospholipids.

5. The method of claim 1, wherein the adjuvant is a cytokine.

6. The method of claim 5, wherein the cytokine is selected from the group consisting of IL-2, IL-6, IL-7, IL-10, IL-13, and IL-15.

7. The method of claim 1, wherein the adjuvant is a virus.

8. The method of claim 1, further comprising a step of treating the patient with chemotherapy at a time prior to introducing the immature dendritic cells and adjuvant into the tumor tissue.

9. The method of claim 1, further comprising a step of treating the patient with radiation therapy at a time prior to introducing the immature dendritic cells and adjuvant into the tumor tissue.

10. The method of claim 1, further comprising a step of treating the patient with anti T-cell monoclonal antibodies at a time prior to introducing the immature dendritic cells and adjuvant into the tumor tissue.

11. The method of claim 1, comprising the step of evaluating the tumor tissue after fourteen days dating from said introductions to determine if reductions of cells have occurred.

12. A method of reduction of tumor cells in tumor tissue comprising the steps of:
treating a tumor of a patient with a chemotherapy regimen,
collecting monocyte cells from the patient,
culturing the monocyte cells with one or a plurality of factors to form immature dendritic cells from the monocyte cells,
introducing the immature dendritic cells into the tumor tissue of the patient, and
introducing an adjuvant into the tumor tissue of the patient.

13. The method of claim 12, wherein the patient is a human.

14. The method of claim 12, wherein the said one or a plurality of factors are selected from the groups consisting of: IL-4 and GM-CFS.

15. The method of claim 12, wherein the adjuvant is selected from the group consisting of: marignase, LCM, agaricus, OK432, BCG, lentinan, Reishi, Sarunokoshikake, TNF, Meshimakobu, Froint's complete or incomplete adjuvant, LPS, fatty acids, and phospholipids.

16. The method of claim 12, wherein the adjuvant is a cytokine.

17. The method of claim 16, wherein the cytokine is selected from the group consisting of IL-2, IL-6, IL-7, IL-10, IL-13, and IL-15.

18. The method of claim 12, wherein the adjuvant is a virus.

19. The method of claim 12, further comprising a step of treating the patient with radiation therapy at a time prior to introducing the immature dendritic cells and adjuvant into the tumor tissue.

20. A method of reduction of tumor cells in tumor tissue comprising the steps of:
treating a tumor of a patient with a radiation therapy regimen,
collecting monocyte cells from the patient,
culturing the monocyte cells with one or a plurality of factors to form immature dendritic cells from the monocyte cells,
introducing the immature dendritic cells into the tumor tissue of the patient, and
introducing an adjuvant into the tumor tissue of the patient.

21. The method of claim 20, wherein the patient is a human.

22. The method of claim 20, wherein the said one or a plurality of factors are selected from the groups consisting of: IL-4 and GM-CFS.

23. The method of claim 20, wherein the adjuvant is selected from the group consisting of: marignase, LCM, agaricus, OK432, BCG, lentinan, Reishi, Sarunokoshikake, TNF, Meshimakobu, Froint's complete or incomplete adjuvant, LPS, fatty acids, and phospholipids.

24. The method of claim 20, wherein the adjuvant is a cytokine.

25. The method of claim 24, wherein the cytokine is selected from the group consisting of IL-2, IL-6, IL-7, IL-10, IL-13, and IL-15.

26. The method of claim 20, wherein the adjuvant is a virus.

27. The method of claim 20, further comprising a step of treating the patient with chemotherapy at a time prior to introducing the immature dendritic cells and adjuvant into the tumor tissue.

28. A cancer vaccine precursor which when introduced into tumor tissue combines with an antigen for the reduction of a tumor in a patient comprising:
immature dendritic cells derived from monocytes collected from the patient,
an adjuvant, and
antigens from the tumor in the patient.

29. The vaccine of claim 28, wherein the patient is a human.

30. The vaccine of claim 28, wherein the one or a multiplicity of factors are selected from the groups consisting of: IL-4 and GM-CFS.

31. The vaccine of claim 28, wherein the adjuvant is selected from the group consisting of: marignase, LCM, agaricus, OK432, BCG, lentinan, Reishi. Sarunokoshikake, TNF, Meshimakobu, Froint's complete or incomplete adjuvant, LPS, fatty acids, and phospholipids.

32. The vaccine of claim 28, wherein the adjuvant is a cytokine.

33. The vaccine of claim 32, wherein the cytokine is selected from the group consisting of IL-2, IL-6, IL-7, IL-10, IL-13, and IL-15.
